(19) United States
(12) Patent Application Publication
(10) Pub. No.: US 2023/0165572 A1
(43) Pub. Date: Jun. 1, 2023

(54) UNIFIED METHOD SWEAT SAMPLE COLLECTOR

(71) Applicant: University Of Cincinnati, Cincinnati, OH (US)

(72) Inventor: Jason C. Heikenfeld, Cincinnati, OH (US)

(21) Appl. No.: 17/995,436

(22) PCT Filed: Apr. 8, 2021
(86) PCT No.: PCT/US2021/026362

§ 371 Date: Oct. 4, 2022

Related U.S. Application Data
(60) Provisional application No. 63/007,555, filed on Apr. 9, 2020.

(51) Int. Cl. A61B 10/00; A61L 31/02; A61L 31/16

(52) U.S. Cl. CPC A61B 10/0064; A61L 31/028; A61L 31/16; A61N 1/0428

(57) ABSTRACT

Described are devices and methods that that include at least one sweat stimulating and collecting material, which can be a sweat stimulating and collecting iontophoresis material. In methods of stimulating and collecting sweat, the method may include (1) stimulating the production of sweat in a subject by delivering at least one sweat-stimulating substance out of a material and into contact with the skin of a subject, and (2) collecting at least a portion of the sweat in the material. Methods in accordance with principles of the present invention can also include transferring the material to a container, removing the material, and analyzing the sweat captured therein.

### CROSS-REFERENCE TO RELATED APPLICATION

This application claims the benefit of the filing date of U.S. Patent Application Ser. No. 63/007,555, filed on Apr. 9, 2020, the disclosure of which is incorporated by reference herein in its entirety.

### BACKGROUND OF THE INVENTION

This section is intended to introduce the reader to various aspects of art that may be related to various aspects of the present invention, which are described and/or claimed below. This discussion is believed to be helpful in providing the reader with background information to facilitate a better understanding of various aspects of the present invention. Accordingly, it should be understood that these statements are to be read in this light, and not as admissions of prior art.

Diagnostic sample collection has advanced significantly, now including devices and tools for minimum-pain blood collection and dried blood spot analysis, and even pain-free microneedle devices to collect tens or hundreds of μLs of blood. Urine and saliva diagnostics have arguably seen less advances, but are still utilized for a wide range of applications. For many applications, sweat can arguably provide superior diagnostic value to saliva and urine, and unlike blood can be accessed truly non-invasively. However, sweat collection tools to date have arguably been too cumbersome to see increased diagnostic use, and until just recently sweat has been poorly understood as a diagnostic fluid. Sweat is known to be useful for small hydrophobic molecule detection (drugs, hormones, etc.) with strong blood correlation, and to even be used for viral antibody detection. The entry route for proteins into sweat is non-selective. And so, even though those proteins are dilute, the ratios of many, if not most, proteins will correlate with ratios in blood. This allows, for example, use of sweat to quantitatively measure IL-6 or troponin, by normalizing it to albumin (which does not change in blood concentration). If a simpler sweat sample collector and method could be developed, sweat could see significantly increased diagnostic value. It is further apparent, that with the advent of inexpensive iontophoresis patches, such as the Iomed Companion 80 (˜$5-6 each retail), the costs of sweat stimulation could be low enough to allow sweat sampling in any user state (e.g. the user does not need to be actively perspiring).

It is worthwhile to further elaborate on several non-limiting examples for the specific utility that sweat sample collection could provide compared to other biofluids. First, sweat could arguably provide the simplest non-invasive biofluid to collect. Saliva collection is prone to contamination and requires careful collection, and urine requires a manual transfer and potential mess for cleanup. Simplicity of collection is particularly important for those with mental illness, the elderly, and young children. Second, sweat is the safest biofluid to collect as it is free of pathogens from the body. For example, sweat does contain diluted viral antibodies that can determine if someone has had, for example, the flu, but has not been shown (for any virus) to contain adequate virus to cause spread of infection. This is simply not the case for saliva, urine, and blood, all of which can easily spread disease. Third, sweat is arguably the least cheatable biofluid for mandatory testing against illicit drug use, drug compliance, disease, readiness for duty or other applications where the sample can be exchanged or altered. For example, a sweat collector can be quickly applied by a certified individual, and the test-subject unable to remove the collector without detection (skin impedance, etc.) until the test is complete and the collector removed by the certified individual. Fourth, sweat can be affected by local skin conditions and therefore has diagnostic value for dermatology. And fifth, sweat has strong value for toxin exposure, and measuring an incident of toxin exposure or even pro-longed toxin exposure and total dosage absorbed into the body.

### SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be explicitly set forth below.

Sweat collectors can be simple and low-cost, but the simplicity and cost can be further improved. Many of the drawbacks and limitations of existing sweat collectors can be resolved by including a sweat stimulation unit that unifies the stimulation and collection into a single method and/or material.

And so, one aspect of the present invention is directed to a sweat collecting device that includes at least one sweat stimulating and collecting material.

Another aspect is directed to a method of stimulating and collecting sweat, the method including (1) stimulating the production of sweat in a subject by delivering at least one sweat-stimulating substance out of a material and into contact with the skin of a subject, and (2) collecting at least a portion of the sweat in the material. In certain embodiments, the material can be an iontophoresis material. Methods in accordance with principles of the present invention can also include transferring the material to a container, removing the material, and analyzing the sweat captured therein.

Yet another aspect is directed to a sweat collection system including (1) a sweat collecting device including at least one sweat-stimulating and collecting material, and (2) a container adapted to receive the at least one sweat-stimulating and collecting material. In certain embodiments the material and/or the container may contain at least one preservative.

Embodiments of the disclosed invention under these and other aspects are directed to materials and methods that create unified method and material sweat sample collectors.

### BRIEF DESCRIPTION OF THE DRAWINGS

The objects and advantages of the disclosed invention will be further appreciated in light of the following detailed descriptions and drawings in which:

FIGS. 1A-1B are cross-sectional views of a device in accordance with principles of the present invention.

FIG. 1C is a cross sectional view showing a portion of the device of FIGS. 1A and 1B positioned within a container.

FIG. 1D is a cross sectional view showing a portion of the device of FIGS. 1A and 1B positioned within an alternate embodiment of a container.

FIG. 2 is a cross-sectional view of another embodiment of a device in accordance with principles of the present invention.

FIG. 3 is a cross-sectional view of yet another embodiment of a device in accordance with principles of the present invention.

FIG. 4 is a cross-sectional view of yet another embodiment of a device in accordance with principles of the present invention.

### DEFINITIONS

As used herein, a sweat stimulating and collecting iontophoresis material, or sweat stimulating and collecting material, means any material that can chemically stimulate sweat and which has a capacity to absorb the sweat it creates by stimulation. For this reason, sweat stimulating and collecting iontophoresis material is different than a standard simulating material, e.g., gels such as agar and pilocarpine, because these materials are typically fabricated in a manner such that they are fully hydrated (whereas the sweat stimulating and collecting material or sweat stimulating and collecting iontophoresis material herein can be less than fully hydrated). In the present invention, the material (e.g., gel), components in it, or a material or component next to it, has capacity to absorb sweat. For example, an agar gel could be partly dried before use and able to reswell with sweat. For example, a small percent of polyacrylamide granuals could be added to an agar gel as it is formed to increase its capacity to absorb sweat. These are non-limiting examples.

### DETAILED DESCRIPTION OF THE INVENTION

One skilled in the art will recognize that the various embodiments may be practiced without one or more of the specific details described herein, or with other replacement and/or additional methods, materials, or components. In other instances, well-known structures, materials, or operations are not shown or described in detail herein to avoid obscuring aspects of various embodiments of the invention. Similarly, for purposes of explanation, specific numbers, materials, and configurations are set forth herein in order to provide a thorough understanding of the invention. Furthermore, it is understood that the various embodiments shown in the figures are illustrative representations and are not necessarily drawn to scale. Further, “a component” may be representative of one or more components and, thus, may be used herein to mean “at least one.” Sweat stimulation, or sweat activation, can be achieved by known methods. For example, sweat stimulation can be achieved drugs such as carbachol, methacholine or pilocarpine, other stimulants, and by dermal introduction of such drugs using iontophoresis, by sudo-motor-axon reflex sweating, or by other means. The disclosure applies to devices with various configurations including patches, bands, straps, portions of clothing, wearables, or any suitable mechanism that reliably brings sweat stimulating and sweat collecting into a unified device. Some embodiments use adhesives to hold the device near the skin, but devices may also be secured by another suitable mechanism, such as a strap or helmet suspension. Certain embodiments of the disclosed invention show sub-components that may require additional obvious sub-components for use of the device in various applications (such as a battery), and for purpose of brevity and focus on inventive aspects are not explicitly shown in the diagrams or described in the embodiments of the present disclosure.

As described above, one aspect of the present invention is directed to a sweat collecting device that includes at least one sweat stimulating and collecting material. In certain embodiments, this material can be a sweat stimulating and collecting iontophoresis material. Below, various embodiments of the invention may refer to an “iontophoresis material” or “sweat-stimulating and collecting iontophoresis material.” Those skilled in the art will recognize that other embodiments may use a material other than an iontophoresis material.

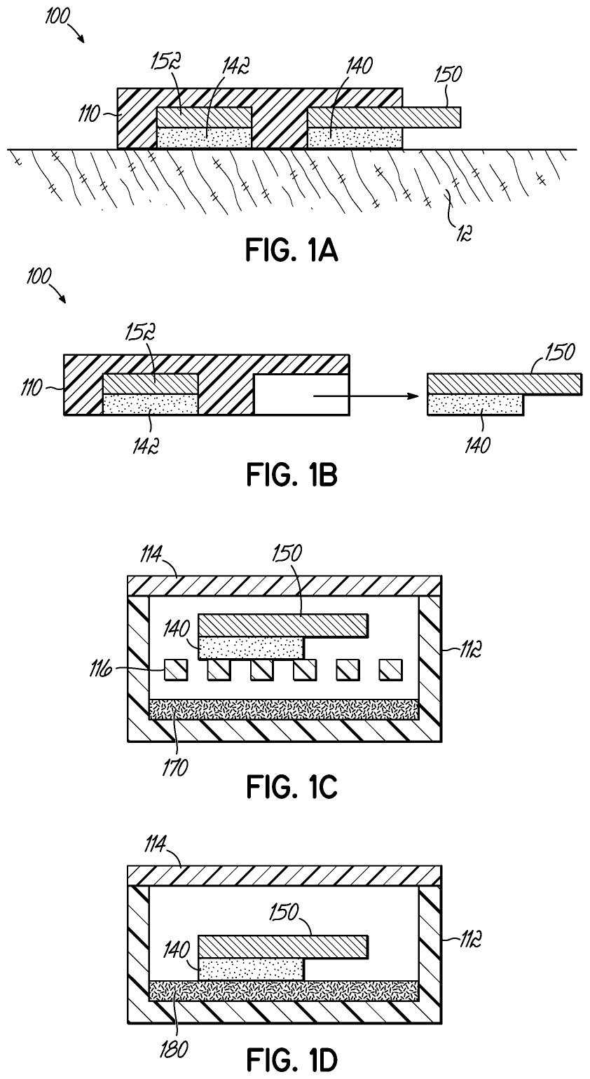

Turning now to the Figures: With respect to FIG. 1A, a device 100 is placed on skin 12 and includes at least one housing material 110, iontophoresis electrodes 150, 152 and iontophoresis material 140, 142 to provide wet skin contact. Housing 110 can be plastic and held to the skin with a strap or be an adhesive and polymer materials similar to that used commercially (e.g. Iomed Companion 80, commercially available from IOMED of Barcelona, Spain). Electrodes 150, 152 can be any suitable material such as carbon rubber or polymer, metal mesh, metal films, or other suitable conducting materials, as known by those skilled in the art of iontophoresis. Iontophoresis material 140, 142 can be agar gel, skin adhesives, or other materials known by those skilled in the art of iontophoresis. At least one iontophoresis material contains a sweat stimulant. Iontophoresis material 140 may contain 1% pilocarpine hydrochloride as the positive pole in iontophoresis, and material 142 may be the negative pole with 0.9% NaCl solution. Based on retail costs for disposable iontophoresis devices, the entire device retail cost circa 2020 could be $5-6 each or even lower. Alternately, material 140 could contain a sweat stimulant that does not require iontophoresis, such as carbachol as the stimulant and propylene glycol as a skin permeation enhancer. Further, as described above, the material (or iontophoresis material) operates both in the stimulation of sweat and serves to collect sweat that is stimulated (that sweat subsequently being analyzed). This is different than a standard simulating material, because standard materials (e.g., gels such as agar and pilocarpine) are typically fabricated in a manner such that they are fully hydrated (whereas the sweat stimulating and collecting material, or sweat stimulating and collecting iontophoresis material herein can be less than fully hydrated). For example, in certain embodiments, the at least one sweat stimulating and collecting material has a degree of hydration such that the volume of the at least one sweat stimulating and collecting material is at least one of 1%, 5%, 10%, 20%, 50%, or 90% less than the volume of the at least one sweat stimulating and collecting material in a fully hydrated state.

With further respect to FIG. 1A, the device 100 is used to stimulate sweat, and the sweat collected by iontophoresis material 140. With respect to FIG. 1B, electrode 150 and material 140 are then removed from the device. Electrode 150 and material 140 could then be placed into a solution to diffuse out sweat solutes which then can be analyzed using equipment or assays such as ELISA. Alternately, as shown in FIG. 1C, electrode 150 and material 140 could be transferred to a metal, glass, or polymer, or other suitable container 112, 114 for storage or transport to a lab. Alternately, entire device 100 could be transferred to a metal, glass, or polymer, or other suitable container 112, 114 for storage or transport to a lab. Container 112, 114 may contain a separator 116 such as a plastic mesh or other suitable material to separate material 140 from a desiccant 170 which at least partially removes water from material 140 to reduce sweat sample degradation during transport to a lab for analysis or for storage. Alternately only material 140 could be transferred into container 112, 114. Alternately, electrode 150 and material 140 could be placed with electrode 150 facing away from desiccant 170 to prevent material 140 from directly touching desiccant 170. The device 100 materials can be shaped such that materials 150 or 140 can only be inserted into container 112, 114 and container 112, 114 sealed such that desiccant 170 does not directly touch material 140 (not shown). Alternately a desiccant, drying, or warming material could be provided in the device that does touch material 140 (not shown), for example by using a forward osmosis membrane such as WaterDrop F₂O™ (Fluid Technology Solutions Inc., Oregon USA) which can remove water from material 140 but which blocks small molecule solutes such that they would retain in material 140.

With further respect to FIGS. 1A-1C, an informative calculation can be used to further teach an example for the present invention. Assume a clean skin surface that is cleaned with water as well to reduce ionic impurities that can interfere with iontophoresis. Assume a full pilocarpine dose for sweat stimulation of 80 μg/cm², an iontophoresis material 140 of agar hydrogel with 1% pilocarpine by weight. Assume the agar hydrogel with pilocarpine 140 had been dried to 50% of its original fabricated volume to allow it to wick up sweat. Further assume 70% efficiency for the iontophoresis to deliver pilocarpine into the skin to stimulate sweat (due to other ionic impurities in the gel 140 and from the skin 12). Assume each 1 μm of the hydrogel 140 represents approximately 100 nL/cm² of fluid volume which is equivalent to 100 μg/cm² of mass of fluid for each μm thickness of hydrogel. Therefore, if the pilocarpine in the hydrogel 140 was 1000 μm (1 mm) thick the hydrogel 140 would have a total mass of 1000*100 μg/cm²=1E5 μg/cm². Pilocarpine represents 1% of this mass (as stated above) or a total of 1000 μg of Pilocarpine, and therefore the required dose of pilocarpine (˜80 μg/cm2) only represents 8% of the total pilocarpine in the hydrogel 140. Therefore this 1 mm thick hydrogel 140 is adequately thick to provide repeatable dosage of pilocarpine by iontophoresis. Next, assume sweat generates 5 nL/min/gland for 30 minutes which translates to 5E-9 L/min/gland * 100 glands/cm2 * 30 minutes=15 μL/cm² of sweat. The 1 mm hydrogel represents an initial volume of 100 μL/cm², such that the sweat and its analytes absorbed into the hydrogel is diluted by only 15% by mixing with the hydrogel 140. If less sweat dilution was desired, the pilocarpine could be 10% by weight in the hydrogel 140 and therefore the hydrogel only need be 100 μm thick or 10 μL/cm² total volume. Assuming the hydrogel 140 was able to absorb the collected sweat, for this case the sweat dilution would be only ˜40%. These calculations indicate that the iontophoresis material 140 can also act as an absorber of sweat and its analytes with minimum dilution of the sweat. Generally thinner iontophoresis material 140 would also promote faster diffusion of sweat analytes out of the iontophoresis material 140 when it was time to analyze the sweat with equipment or assays. The present invention therefore includes materials 140 that are less than 0.5, 0.2, 0.1, 0.05, or 0.02 cm in thickness resulting in dilution of sweat samples that are <90%, <50%, <20%, <10%, or <5%.

With further respect to FIG. 1C, in order to at least partially preserve the sweat stored in material 140 and its analytes, a desiccant 170 such as a hydrogel, paper, silica, salt, or other suitable desiccant can be used. The material 140 and desiccant 170 can be designed for fairly predictable dehydration of the material 140 based on osmotic, wicking, and other forces that control how much water would remain in the material 140. Water reduction will decrease the activity of bacteria, enzymes, and other solutes that could degrade analytes in the collected sweat. In addition, the material 140 can include, or have added to it after sweat collection and before storage, at least one preservative. If the preservative is added into the material 140 before sweat stimulation (iontophoresis), then ideally such preservative is non-ionic and skin safe. For example, such a preservative could be a parabens, which is a family of esters based on parahydroxybenzoic acid that show strong antimicrobial properties and they are used in cosmetic products as preservative ingredients. Alternately, the preservative could be charged negatively such that it would not be driven into the skin 12 during iontophoresis.

As described above, the device 100 is used to stimulate sweat, and the sweat is collected by iontophoresis material 140. With respect to FIG. 1B, electrode 150 and material 140 may then be removed from the device. As shown in FIG. 1D, electrode 150 and material 140 could be transferred to a container 112, 114 for storage or transport to a lab, for example. Container may be solid (such as made from metal, glass, or polymer) or a flexible pouch. In order to at least partially preserve the sweat stored in material 140 and its analytes, the container may include at least one preservative 180. Preservative 180 may be coated on or impregnated in at least one interior surface of the container, such that material 140 contacts preservative 180 when material 140 (or material 140 and electrode 150) are placed in container 112, 114. Alternatively, preservative may be present within interior of container without being specifically coated on or impregnated in any interior surface.

With further respect to FIGS. 1A-1C, in order to allow quantitative measurement of solutes in sweat, the volume of the collected sweat sample must be known or determined. As a non-limiting example, materials 140 and 150 could have a measured or predicted weight at the time of their manufacturing. If this weight were measured, it could, for example, be labeled on the material 150 (such as the backside of material 150) as weight or as a barcode. Similarly, the weight of materials 114, 112, 116, and 170 could be measured or predicted. Therefore, before material 140 is removed to analyze the sweat and its solutes, the entire device 100 be weighed and any additional weight beyond the original measured or predicted weight would be due to absorption of sweat. This is then translatable to a volume of sweat, and a precise calculation of the amount of dilution of the sweat into material 140 is easily calculated.

With respect to the present invention, in most cases the sweat solutes will be extracted to be analyzed. In many assays, the biofluid sample is mixed with another fluid before the assay is performed. As a non-limiting example, material 140 could be placed into a volume of assay fluid (not shown) to allow sweat solutes in material 140 to diffuse into the assay fluid. A question then exists as to what solutes are recoverable and what are not, especially if the material 140 is partially dried which not only causes some proteins to become insoluble but also changes the salinity and pH of the solution in which the solutes reside. An instructive analogy for sample recovery is provided by the more difficult task of dried blood spot sample recovery and analysis. As this analogy is discussed, it is important to note that the most meaningful solutes in sweat from a diagnostic perspective are typically small molecules that are easily recovered even from dried solutions, and which have fast diffusion times from one material into another due to their small size. Clinical testing using whole blood dried on filter paper specimens has been performed since the 1960s. Dried blood spot testing has been used extensively for newborn screening of preventable diseases, therapeutic drug monitoring, pharmacokinetics, testing for drugs of abuse, and measurement of analyte classes such as small molecules, proteins, and nucleic acids. In summary, dried blood spot testing works, and is arguably more difficult than the sample recovery for sweat with the present invention.

With reference to Figures of the present invention, the device could include Ag/AgCl electrodes to measure Cl— content for Cystic fibrosis testing. Because pilocarpine nitrate does not contain Cl— ions, sweat Cl— could be detected using a simple Ag/Cl electrode which could also be the iontophoresis electrode, or another type of sensor. Therefore, the device could be to collect a sample for lab analysis and do a real time or point-of-care type measurement.

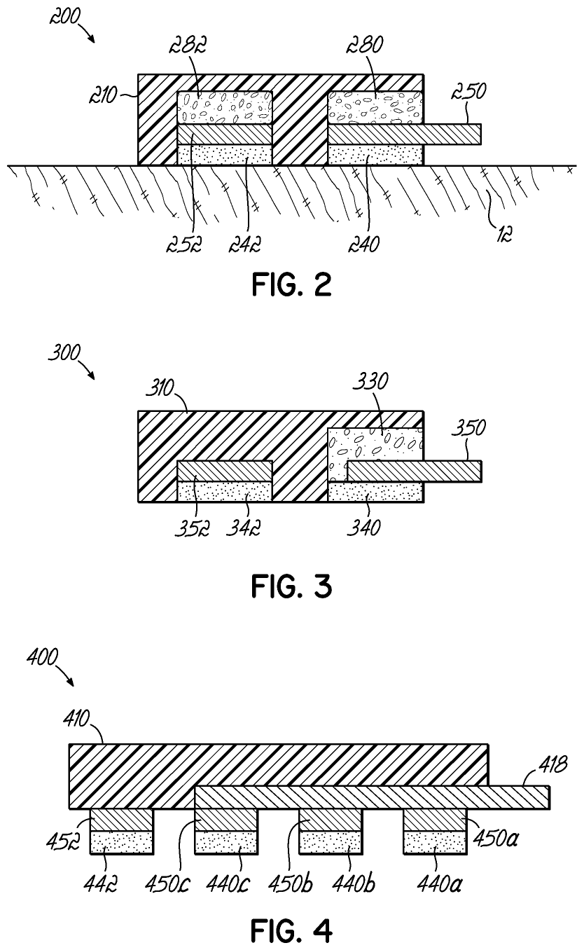

With reference now to FIG. 2, where like numerals refer to like features, a device 200 further includes at least one pressure-providing material 280, 282 such as a sponge, spring, bag of gel or air, or other suitable material that promotes proper coupling of material 240 to skin 12. This can be particularly helpful because material 240 may be very thin, and a conformal application to skin 12 therefore more difficult. Device 200 includes at least one housing material 210, iontophoresis electrodes 250, 252 and iontophoresis material 240, 242. Housing 210 (like that described with respect to FIG. 1A) can be plastic and held to the skin with a strap or be an adhesive and polymer materials similar to that used commercially (e.g. Iomed Companion 80). Electrodes 250, 252 can be any suitable material such as carbon rubber or polymer, metal mesh, metal films, or other suitable conducting materials, as known by those skilled in the art of iontophoresis. Iontophoresis material 240, 242 can be agar gel, skin adhesives, or other materials known by those skilled in the art of iontophoresis. In one embodiment, iontophoresis material 240 may contain 1% pilocarpine hydrochloride as the positive pole in iontophoresis, and material 242 may be the negative pole with 0.9% NaCl solution. At least one iontophoresis material contains a sweat stimulant (as described above). Alternately, material 240 could contain a sweat stimulant that does not require iontophoresis, such as carbachol as the stimulant and propylene glycol as a skin permeation enhancer. Further, as described above, the material (or iontophoresis material) operates both in the stimulation of sweat and serves to collect sweat that is stimulated (that sweat subsequently being analyzed). In certain embodiments, the at least one sweat stimulating and collecting material has a degree of hydration such that the volume of the at least one sweat stimulating and collecting material is at least one of 1%, 5%, 10%, 20%, 50%, or 90% less than the volume of the at least one sweat stimulating and collecting material in a fully hydrated state.

With reference to FIG. 3, where like numerals refer to like features, a device 300 further includes at least one wicking component 330 that receives fluid and sweat from material 340. For example, material 340 could be a wicking material such as agar with a very strong wicking pressure and a fixed volume, whereas material 330 could be a capillary tube, matrix of micro beads, or other material with a fairly homogenous wicking (capillary) pressure that is less than that of material 340 such that, before use of device 300, material 330 is primarily devoid of fluid, but as sweat is collected by device 300 and exceeds the volume or wicking capacity of material 340 the excess fluid (mixed sweat plus original fluid in 340) would be received by material 330. Material 330 and/or material 340 could then be removed from device 300 and sent for analysis of sweat solutes.

Still referring to FIG. 3, device 300 includes at least one housing material 310, iontophoresis electrodes 350, 352 and iontophoresis material 340, 342. Housing 310 (like that described with respect to FIG. 1A) can be plastic and held to the skin with a strap or be an adhesive and polymer materials similar to that used commercially (e.g. Iomed Companion 80). Electrodes 350, 352 can be any suitable material such as carbon rubber or polymer, metal mesh, metal films, or other suitable conducting materials, as known by those skilled in the art of iontophoresis. Iontophoresis material 340, 342 can be agar gel, skin adhesives, or other materials known by those skilled in the art of iontophoresis. In one embodiment, iontophoresis material 340 may contain 1% pilocarpine hydrochloride as the positive pole in iontophoresis, and material 342 may be the negative pole with 0.9% NaCl solution. At least one iontophoresis material contains a sweat stimulant (as described above). Alternately, material 340 could contain a sweat stimulant that does not require iontophoresis, such as carbachol as the stimulant and propylene glycol as a skin permeation enhancer. Further, as described above, the material (or iontophoresis material) operates both in the stimulation of sweat and serves to collect sweat that is stimulated (that sweat subsequently being analyzed). In certain embodiments, the at least one sweat stimulating and collecting material has a degree of hydration such that the volume of the at least one sweat stimulating and collecting material is at least one of 1%, 5%, 10%, 20%, 50%, or 90% less than the volume of the at least one sweat stimulating and collecting material in a fully hydrated state.

With further reference to FIG. 3, one advantage of the present invention, (as illustrated in FIG. 1), is that a desiccant can remove water from material 140, 340, and material 330. This effectively concentrates the sweat sample, which can simplify the analysis of the sample in the lab for analytes. A remaining challenge, however, is recovery of the sweat sample from the dehydrated material 140 without material 140 retaining too much water (which in turn causes loss of sample). In certain embodiments, the material 140, 340 and or material 330 can have a weak wicking strength by being made of sponge or other material with pores that are sub mm to micrometer and sample extracted by spinning the sample in a centrifuge. In certain embodiments, the material 140, 340 and or material 330 can be a hydrogel or other material that when dehydrated does not swell or quickly swell to its rehydrated volume. Polyacrylamide, agar, and other hydrogels when dried can be designed to slowly reswell over time (10's minutes to hours or days), and analytes of interest in a sample could quickly (1's minutes to hours) diffuse out of the material 140, 340, 330 into an extraction solution such as buffer solution before materials 140, 340, 330 significantly swell and cause loss of sample. In certain embodiments, the material 140, 340 and or material 330 can be a material that when dried reswells due to osmotic pressure (polyacryalamide being an example). When the sample is extracted from material 140, 340 and or material 330 an extraction solution could then have strong ionic or osmotic strength, to allow analytes to diffuse out of sample but to suppress swelling of material 140, 340 and or material 330. Other options may exists as well, such as placing the material 140, 340 and or material 330 under physical confinement during sample extraction such that it cannot swell. Generally, at least 20%, 50% or 90% of sample can be extracted from materials 140, 340, 330.

With reference to FIG. 4, where like numerals refer to like features, a device 400 includes at least one housing material 410, a plurality of iontophoresis electrodes 450a, 450b, 450c and a plurality of iontophoresis materials 440a, 440b, 440c, such that a plurality of iontophoresis events can be administered and a plurality of sweat samples collected over time. Device 400 also includes iontophoresis electrode 452 and iontophoresis material 442. In one embodiment, iontophoresis materials 440a, 440b, 440c may contain 1% pilocarpine hydrochloride as the positive pole in iontophoresis, and material 442 may be the negative pole with 0.9% NaCl solution. Materials 440a, 440b, 440c could remain in skin contact or be actuated in and out of skin contact mechanically (such as with a speaker coil actuator) to preserve the integrity of the sweat stimulant and/or the collected sweat sample. A single material 418 could allow all materials with sweat samples to be easily removed from device and sent for analysis. For example, if six such sweat samples were collected, a drug pharmacokinetic curve could be collected and analyzed non-invasively for drugs in sweat that have strong sweat-blood correlation. In this example, the entire device 400 could also be sealed with a desiccant and sent to a lab for analysis, using methods as previously taught for the present invention.

The embodiments of the present invention recited herein are intended to be merely exemplary and those skilled in the art will be able to make numerous variations and modifications to it without departing from the spirit of the present invention. Notwithstanding the above, certain variations and modifications, while producing less than optimal results, may still produce satisfactory results. All such variations and modifications are intended to be within the scope of the present invention as defined by the claims appended hereto.

What is claimed is:

1. A sweat collecting device comprising at least one sweat stimulating and collecting material.

2. The device of claim 1, wherein the at least one sweat stimulating and collecting material includes a sweat stimulant.

3. The device of claim 2, wherein the sweat stimulant includes at least one of pilocarpine, methacholine, or carbachol.

4. The device of claim 2, wherein the at least one sweat stimulating and collecting material further includes at least one preservative.

5. The device of claim 4, wherein the at least one preservative is non-ionic and skin safe.

6. The device of claim 1, further comprising an iontophoresis electrode associated with the at least one sweat stimulating and collecting material.

7. The device of claim 6, further comprising a second iontophoresis electrode associated with a sweat stimulating material.

8. The device of claim 7, wherein the sweat stimulating material may include sodium chloride.

9. The device of claim 7, further comprising a housing, wherein the iontophoresis electrode, second iontophoresis electrode, at least one sweat stimulating and collecting material, and sweat stimulating iontophoresis material are disposed at least partially within the housing.

10. The device of claim 9, wherein the at least one sweat stimulating and collecting material is adapted to be removable from the housing.

11. The device of claim 1, further comprising at least one pressure-providing material associated with the at least one sweat stimulating and collecting material.

12. The device of claim 1, further comprising at least one wicking material associated with at least one sweat stimulating and collecting material.

13. The device of claim 12, wherein the at least one wicking material has a capillary pressure less than a capillary pressure of the at least one sweat stimulating and collecting material.

14. The device of claim 1, wherein the at least one sweat stimulating and collecting iontophoresis material has a degree of hydration such that the volume of the at least one sweat stimulating and collecting material is at least one of 1%, 5%, 10%, 20%, 50%, 90% or less than the volume of the at least one sweat stimulating and collecting material in a fully hydrated state.

15. The device of claim 1, further comprising at least one storage container.

16. The device of claim 15, further comprising at least one desiccant in the storage container.

17. The device of claim 15 wherein said desiccant and said stimulating and collecting material are physically separated from direct contact by at least one material.

18. The device of claim 1, where said storage container has a known or measured weight and said stimulating and collecting material has a known or measured weight, and both weights can be used to determine the amount of sweat collected by the device.

19. The device of claim 1, wherein said stimulating and collecting material is at least one of less than 0.5, 0.2, 0.1, 0.05, 0.02 cm in thickness, resulting in dilution of sweat sample that are less than at least one of <90%, <50%, <20%, <10%, <5%.

20. The device of claim 15, further comprising at least one preservative in the storage container.

21. The device of claim 21, wherein said preservative and said stimulating and collecting material are in direct contact when said stimulating and collecting material is placed within said storage container.

22. The device of claim 15, further comprising at least one forward osmosis component that removes water from said stimulating and collecting material while retaining one or more types of solutes in the stimulating and collecting material.

23. The device of claim 1, further comprising an extraction solution to remove collected sweat sample from said stimulating and collecting material, wherein the extraction of sweat sample is at least one greater than 20%, 50%, or 90% of the collected sample.

24. The device of claim 15, further comprising an extraction solution to remove collected sweat sample from said stimulating and collecting material, wherein the stimulating and collecting material is material that when placed in the storage container dries and shrinks in volume, and the sweat stimulating and collecting material does not fully rehydrate and swell in volume when placed in the extraction solution.

25. The device of claim 1, wherein the at least one sweat stimulating and collecting material includes a sweat stimulant and a fluid to enhance diffusion of the sweat stimulant into the skin.

26. The device of claim 25, wherein the sweat stimulant is carbachol.

27. A method of collection a sweat sample comprising, stimulating sweat with stimulating and collecting iontophoresis material, collecting the sweat with the same stimulating and collecting iontophoresis material, transferring the stimulating and collecting iontophoresis material to a storage container that preserves the collected sweat sample, removing the stimulating and collecting iontophoresis material and analyzing the collected sweat captured therein.

28. The method of claim 27, wherein the weights of the stimulating and collecting iontophoresis material and of the storage container are measured or know such that the quantity of sweat collected is known and therefore the absolute concentration of analyte in the collected sweat can be measured.

29. A method of stimulating and collecting sweat, the method comprising:
stimulating the production of sweat in a subject by delivering at least one sweat-stimulating substance out of a material and into contact with the skin of a subject; and
collecting at least a portion of the sweat in the material.

30. The method of claim 29, wherein delivering at least one sweat-stimulating component further comprises use of an electric current to transport said at least one sweat-stimulating substance to the skin.

31. The method of claim 29, further comprising the use of a fluid to enhance diffusion of the sweat-stimulating substance into the skin.

32. The method of claim 31, wherein the fluid is included in said material.

33. The method of claim 29, further comprising removing sweat solutes from said material.

34. The method of claim 33, wherein removing sweat solutes from said material further comprises use of an extraction solution to remove collected sweat sample from said stimulating and collecting material, wherein the stimulating and collecting material is material that when placed in the storage container dries and shrinks in volume, and the sweat stimulating and collecting material does not fully rehydrate and swell in volume when placed in the extraction solution.

35. The method of claim 33, wherein removing sweat solutes from said material further comprises spinning said material in a centrifuge.

36. The method of claim 33, wherein removing sweat solutes further comprises placing the material in a solution to cause diffusion of the sweat solutes out of the material.

37. The method of claim 36, further comprising removing the material from a device of which it is one component prior to placing the material in the solution.

38. The method of claim 29, further comprising removing the material from a device of which it is one component, and placing the material into a container for storage or transport.

39. The method of claim 38, wherein the material further includes water, and wherein the method further comprises removing at least a portion of the water from the material.

40. The method of claim 39, wherein removing at least a portion of the water further comprises use of a desiccant.

41. The method of claim 40, further comprising preventing contact between the material and the desiccant.

42. The method of claim 29, further comprising analyzing analytes in the sweat, and diagnosing a condition or characteristic of the subject based on the analysis.

43. The method of claim 29, further comprising reducing or preventing degradation of any analytes in the sweat.

44. The method of claim 43, wherein reducing or preventing degradation of any analytes occurs via use of a preservative in the material.

45. The method of claim 44, further comprising adding the preservative after collecting the sweat in the material.

46. The method of claim 29, wherein the material is a hydrogel.

47. The method of claim 46, wherein the hydrogel is an agar hydrogel including 1% pilocarpine by weight.

48. The method of claim 47, wherein the material is dried to 50% of its original fabricated volume.

49. The method of claim 47, wherein the agar hydrogel has a total mass of 1E5 μg/cm2.

50. The method of claim 47, wherein stimulating the production of sweat in a subject comprises the use of 8%-10% of the total pilocarpine in the material.

51. The method of claim 34, wherein the material is capable of reswelling due to osmotic pressure once dried.

52. The method of claim 51, wherein said extraction solution is of an ionic or osmotic strength that allows analytes to diffuse out of said sweat while suppressing swelling of said material.

53. The method of claim 29, wherein stimulating the production of sweat in a subject results in the generation of sweat at a rate of 5 nL/min/gland.

54. The method of claim 53, wherein the generation of sweat occurs for 30 minutes.

55. The method of claim 29, wherein stimulating the production of sweat further comprises delivering at least one sweat-stimulating substance out of a plurality of materials, and wherein collecting at least a portion of the sweat further comprises collecting a separate sweat sample in each of said plurality of materials.

56. The method of claim 55, further comprising placing a device, of which the plurality of materials are components, into a container for storage or transport.

57. The method of claim 56, wherein the container includes a desiccant, and the method further comprises preventing contact between the device and the desiccant.

58. The method of claim 29, wherein the material is an iontophoresis material.

59. A sweat collection system comprising:
a sweat collecting device comprising at least one sweat stimulating and collecting material; and
a container adapted to receive the at least one sweat stimulating and collecting material.

60. The system of claim 59, wherein the at least one sweat stimulating and collecting material is at least one sweat stimulating and collecting iontophoresis material.

61. The system of claim 59, wherein the container further comprises a housing defining an interior chamber having a portion thereof for receiving the at least one sweat stimulating and collecting iontophoresis material.

62. The system of claim 61, further comprising a desiccant disposed within the interior chamber.

63. The system of claim 62, further comprising a separator disposed within the interior chamber and positioned between the desiccant and the portion of the interior chamber for receiving the at least one sweat stimulating and collecting material.

64. The system of claim 59, wherein the container has a known or measured weight and the stimulating and collecting material has a known or measured weight, and both weights can be used to determine the amount of sweat collected by the device.

65. The system of claim 59, wherein the sweat stimulating and collecting material is at least one of less than 0.5, 0.2, 0.1, 0.05, or 0.02 cm in thickness, resulting in a dilution of a sweat sample that is less than at least one of <90%, <50%, <20%, <10%, or <5%.

66. The system of claim 59, further comprising at least one preservative in said container.

67. The device of claim 66, wherein said preservative and said stimulating and collecting material are in direct contact when said stimulating and collecting material is placed within said container.

68. The device of claim 1, further comprising at least one analyte sensor.
